(19) United States
(12) Patent Application Publication
(10) Pub. No.: US 2019/0336471 A1
(43) Pub. Date: Nov. 7, 2019

(54) COMPOSITIONS CONTAINING OMEGA-3 FATTY ACIDS CONCENTRATES, CANNABANOIDS AND L-THEANINE AND USE THEREOF

(71) Applicant: Omax Health, Los Angeles, CA (US)

(72) Inventors: Evan DeMarco, Sacramento, CA (US); Kristin Chadwick, Marina Del Rey, CA (US)

(21) Appl. No.: 16/398,636

(22) Filed: Apr. 30, 2019

Related U.S. Application Data
(60) Provisional application No. 62/665,907, filed on May 2, 2018.

(51) Int. Cl. A61K 31/352; A61K 47/14; A61K 31/198

(52) U.S. Cl. CPC A61K 31/352; A61K 31/198; A61K 47/14

(57) ABSTRACT

The present disclosure relates to compositions that provide anti-anxiety relief and similar benefits and use thereof. In particular, the disclosure provides compositions containing omega-3 fatty acids concentrates, cannabanoids and L-Theanine, and the synergistic effects of these compositions provides multiple health and wellness benefits. The disclosure further provides the use of these compositions in addressing multiple indications for health and wellness, including but not limited to, sleeplessness, anxiety, pain associated with general inflammation, drug addiction, brain injury, and/or alpha wave brain disorder.

### CROSS REFERENCE TO RELATED APPLICATIONS

This application claims priority to U.S. Provisional Patent Application Ser. No. 62/665,907, filed May 2, 2018, of which is herein incorporated by reference in its entirety.

### BACKGROUND

### 1. Field of the Discovery

The present disclosure relates to compositions that provide anti-anxiety relief and similar benefits and use thereof. In particular, the disclosure provides compositions containing omega-3 fatty acids concentrates, cannabanoids and L-Theanine, and the synergistic effects of these compositions provides multiple health and wellness benefits. The disclosure further provides the use of these compositions in addressing multiple indications for health and wellness, including but not limited to, sleeplessness, anxiety, pain associated with general inflammation, drug addiction, brain injury, and/or alpha wave brain disorder.

### 2. Background Information

Polyunsaturated fatty acids (PUFAs) are fatty acids that contain more than one double bond in their backbone. Omega-3 fatty acids, also called ω-3 fatty acids or n-3 fatty acids, are PUFAs. The three most prominent omega-3 fatty acids involved in human physiology are α-linolenic acid (ALA), found in plant oils, and eicosapentaenoic acid (EPA) and docosahexaenoic acid (DHA), both commonly found in marine oils. Omega-3 fatty acids are often referred to as “essential” fatty acids (EFAs) because they are needed for human health but are not sufficiently produced by the body alone.

The omega-3 fatty acids, in particular EPA and DHA, have multiple health benefit in central nervous system, cognitive, cardiovascular, joint, immune and metabolic function. Fatty acids supplements are available. However, conventional over-the-counter omega-3 fatty acid supplements contain relatively impure material and are typically only about 30 wt % omega 3 fatty acids. This low purity leads to inadequate dosing of essential fatty acids unless a large number of dosage units are consumed each day. Additionally research suggests that the EPA:DHA ratio is important for efficacy. Currently, multiple prescription omega-3 fatty acid medications are available, such as OMACOR (containing approximately EPA and DHA in an approximately 3:2 ratio), and VASCEPA (containing essentially “pure” EPA).

A cannabinoid is one of a class of diverse chemical compounds that acts on cannabinoid receptors in cells that alter neurotransmitter release in the brain. Phytocannabinoids are cannabinoids that occur naturally in the cannabis plant. The most notable cannabinoid is the phytocannabinoid tetrahydrocannabinol (THC), the primary psychoactive compound in cannabis. Cannabidiol (CBD) is another major constituent of the plant, and does not appear to have any psychoactive effects such as those caused by tetrahydrocannabinol (THC).

Theanine, also known as L-γ-glutamylethylamide and N⁵-ethyl-L-glutamine, is an amino acid analogue of the proteinogenic amino acids L-Glutamate and L-glutamine and is found primarily in particular plant and fungal species. It was discovered as a constituent of green tea in 1949 and in 1950 was isolated from gyokuro leaves. L-theanine significantly increases activity in the alpha frequency band which indicates that it relaxes the mind without inducing drowsiness. Theanine has been studied for its potential ability to reduce mental and physical stress, improve cognition, and boost mood and cognitive performance in a synergistic manner with caffeine. The name “theanine” without a prefix generally implies the enantiomer L-theanine, which is the form found in tea leaves and as a dietary supplement ingredient.

Over the past several decades, various studies have been centered on the effect of omega-3 fatty acids, phytocannabanoids, or L-Theanine alone. However, these studies are not directed to the synergistic benefits of omega-3 fatty acids, phytocannabanoids and L-theanine.

It is therefore advantageous to provide a composition of omega-3 fatty acids, phytocannabanoids and L-theanine with synergistic effects for multiple health and wellness benefits.

### SUMMARY

The present disclosure relates to novel compositions containing omega-3 fatty acids, phytocannabanoids and L-theanine. In particular, the disclosure provides a composition of omega-3 fatty acids concentrates containing EPA and DHA, cannabanoids, and L-theanine.

In one aspect, the disclosure provides a composition of omega-3 fatty acids concentrates containing EPA and DHA, phytocannabanoids, and L-theanine, wherein the EPA to DHA ratio is in the range of 3.5-7:1.

In another embodiment, the disclosure provides a composition of omega-3 fatty acids concentrates (containing 30-99 wt % of omega-3 fatty acids), phytocannabanoids, and L-theanine.

In another embodiment, the disclosure provides a composition of omega-3 fatty acids concentrates containing 30-99 wt % of omega-3 fatty acids, phytocannabanoids, and L-theanine, wherein the omega-3 fatty acids concentrates contains EPA to DHA in a ratio of 3.5-7:1.

In another embodiment, the disclosure provides a composition of omega-3 fatty acids concentrates containing EPA and DHA, phytocannabanoids, and L-theanine, wherein the phytocannabanoids contains cannabidiol (CBD) with less than 0.3 wt % of tetrahydrocannabinol (THC).

In yet another embodiment, the disclosure provides a composition comprising 100-2,000 mg of 30-99 wt % omega-3 fatty acids concentrates, 1-50 mg phytocannabanoids, and 25-500 mg L-theanine.

The present disclosure further provides the use of the compositions containing omega-3 fatty acids, phytocannabanoids and L-theanine in addressing multiple indications for health and wellness, including but not limited to, sleeplessness, anxiety, pain associated with general inflammation, drug addiction, brain injury, and/or alpha wave brain disorder.

### DETAILED DESCRIPTION

The following is a detailed description provided to aid those skilled in the art in practicing the present disclosure. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

The present description provides compositions containing omega-3 fatty acids, phytocannabanoids and L-theanine and use thereof. Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either both of those included limits are also included in the disclosure.

The following terms are used to describe the present disclosure. Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, all numerical values, with or without modification of “about” or “approximately,” are to be understood to take into account experimental error and variations that would be expected by a person having ordinary skill in the art.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

A “dosage form” means a unit of administration of an active agent. Examples of dosage forms include tablets, capsules, particularly gel and liquid capsules, suspensions, liquids, candy and chewable formulations, emulsions, creams, ointments, suppositories, and the like.

The term “effective amount” or “therapeutically effective amount” means an amount effective, when administered to a patient, to provide any therapeutic benefit. A therapeutic benefit may be an amelioration of symptoms, e.g., an amount effective to decrease the symptoms of sleeplessness, anxiety, pain associated with general inflammation, drug addiction, brain injury, and/or alpha wave brain disorder. In certain circumstances a patient may not present symptoms of a condition for which the patient is being treated. A therapeutically effective amount of an active agent may also be an amount sufficient to provide a significant positive effect on any indicium of a disease, disorder, or condition, e.g. an amount sufficient to significantly reduce the frequency and severity of symptoms. A significant effect on an indicium of a disease, disorder, or condition is statistically significant in a standard parametric test of statistical significance, for example Student's T-test, where p≤0.05. An “effective amount or “therapeutically effective amount” of the compositions provided herein may also be an amount of about of the formulation or of any dosage amount approved by a governmental authority such as the U.S. FDA, for use in treatment.

“Efficacy” means the ability of an active agent administered to a patient to produce a therapeutic effect in the patient.

“Gel capsule” means any soft gelatin, liquid-filled capsule that contains a liquid, liquid suspension, solution, gel, or emulsion.

“Liquid capsule” is a capsule with a hard or soft capsule shell filled with a non-solid formulation. The formulation may be for example a liquid, solution, suspension, emulsion or gel.

“Providing” means giving, administering, selling, distributing, transferring (for profit or not), manufacturing, compounding, or dispensing.

“Omega-3 fatty acids concentrates” contains enriched Omega-3 fatty acids over other types of fatty acids from natural resources, such as fish and algal oils. Omega-3 fatty acids concentrates may contain 30-99 wt % of Omega-3 fatty acids. “DHA” is docosahexaenoic acid and “EPA” is eicosapentaenoic acid. The terms EPA and DHA are used to indicate both the triglyceride and esterified forms of these Omega-3 fatty acids unless the triglyceride or esterified form is clearly indicated by the context. DHA and EPA also include pharmaceutically acceptable fatty acid salts.

“Salts” as used herein describes “pharmaceutically acceptable salts” of omega-3 fatty acids and other active agents discussed herein and also includes solvates and hydrates of such active agents. The active agent may be modified by making non-toxic acid or base addition salt thereof. Examples of pharmaceutically acceptable salts include mineral or organic acid addition salts of basic residues such as amines; alkali or organic addition salts of acidic residues; and the like, and combinations comprising one or more of the foregoing salts. The pharmaceutically acceptable salts include non-toxic salts and the quaternary ammonium salts of the active agent. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts include salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparaginate, glutamate, and the like; and combinations comprising one or more of the foregoing salts.

“Cannabinoid” is one of a class of diverse chemical compounds that acts on cannabinoid receptors in cells that alter neurotransmitter release in the brain. Ligands for these receptor proteins include the endocannabinoids (produced naturally in the body by animals), the phytocannabinoids (found in cannabis and some other plants), and synthetic cannabinoids (manufactured artificially). The most notable cannabinoids are the phytocannabinoid tetrahydrocannabinol (THC) and cannabidiol (CBD).

“Full-spectrum phytocannabinoids” refers to when the pure oil extracted from hemp contains all the same cannabinoids and compounds found in the original hemp plant. Unlike isolated or synthetic cannabinoids, full-spectrum hemp oil contains an array of cannabinoids, as well as many essential vitamins and minerals, fatty acids, protein, chlorophyll, fiber, flavonoids, and terpenes.

Cannabis produces CBD-carboxylic acid through the same metabolic pathway as THC, until the next to last step, where CBDA synthase performs catalysis instead of THCA synthase.

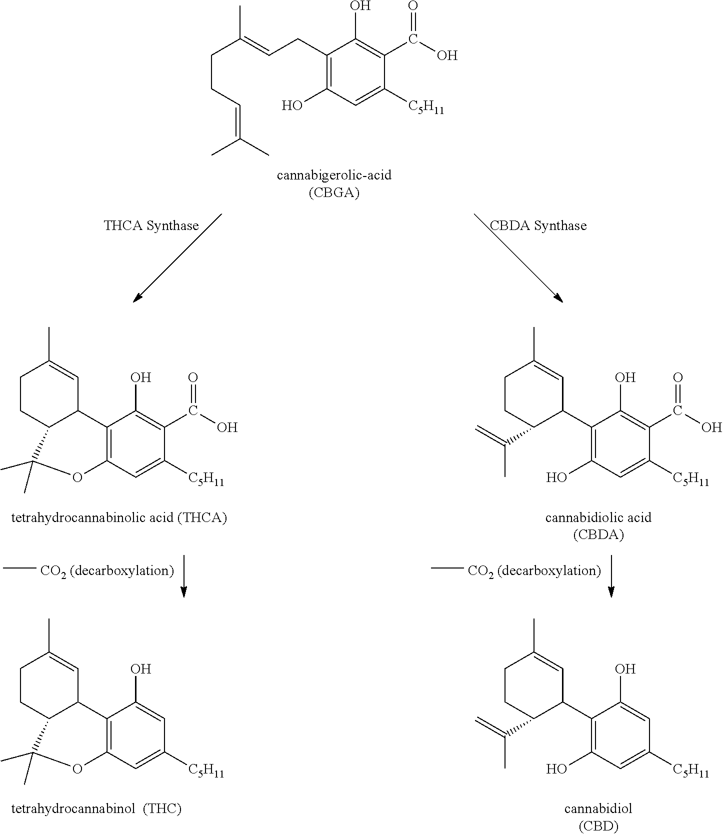

Cannabidiol has very low affinity for the cannabinoid CB₁ and CB₂ receptors but acts as an indirect antagonist of these receptors. It may potentiate THC's effects by increasing CB₁ receptor density or through another CB₁ receptor-related mechanism. Cannabidiol may also extend the duration of the effects of THC via inhibition of the cytochrome P450, CYP3A and CYP2C enzymes.

Cannabidiol has been found to act as an antagonist of GPR55, a G protein-coupled receptor and putative cannabinoid receptor that is expressed in the caudate ucleus and putamen in the brain. It has also been shown to act as a 5-HT1A receptor partial agonist, and this action may be involved in the antidepressant, anxiolytic, and neuroprotective effects of cannabidiol. It is an allosteric modulator of the μ- and δ-opioid receptors as well. Cannabidiol's pharmacological effects have additionally been attributed to PPARγ agonism and intracellular calcium release.

Research suggests that CBD may exert some of its pharmacological action through its inhibition of fatty acid amide hydrolase (FAAH), which may in turn increase the levels of endocannabinoids, such as anandamide, produced by the body. It has also been speculated that some of the metabolites of CBD have pharmacological effects that contribute to the biological activity of CBD.

Recently, cannabidiol has been identified as a novel inverse agonist of the GPR12.

“Theanine,” without prefix, is generally understood to imply the L-(S-)enantiomer, derived from the related proteinogenic L-amino acid glutamic acid. Theanine or L-theanine is an analog of this amino acid, and its primary amide, L-glutamine (also a proteinogenic amino acid). Theanine or L-theanine is a derivative of glutamine that is ethylated on the amide nitrogen (as the name N⁵-ethyl-L-glutamine describes), or alternatively, to the amide formed from ethylamine and L-glutamic acid at its γ-(5-) side chain carboxylic acid group (as the name γ-L-glutamylethylamide describes). Theanine is structurally similar to the excitatory neurotransmitter glutamate, and in accordance, binds to glutamate receptors, though with much lower affinity in comparison, resulting in an anti-stress effect through the inhibition of cortical neuron excitation. Specifically, it binds to ionotropic glutamate receptors in the micromolar range, including the AMPA and kainate receptors and, to a lesser extent, the NMDA receptor. It acts as an antagonist of the former two sites and as an agonist of the latter site.

In addition to the anti-stress effects of L-theanine through competitive action against excitation of glutamate receptor subtypes, neurochemical studies suggest that L-theanine affects emotions by interacting with serotonin, dopamine (DA), and γ-aminobutyric acid (GABA) neurotransmitter levels in the brain.

Both the polyunsaturated fatty acids and the phytocannabinoids impact the endocannabinoid system and the CB₁ and CB₂ receptors located throughout the entire human body, positively impacting inflammation and the inflammatory response. L-theanine has a calming effect on the brain without electing drowsiness, while providing neuroprotection against environmental stress. The synergistic effects of these components provides multiple health and wellness benefits.

U.S. Pat. No. 7,652,068, which is incorporated herein by reference, discloses a pharmaceutical formulation comprising omega-3 fatty acids concentrates with eicosapentaenoic acid (EPA) and docosahexanoic acid (DHA) in a weight to weight ratio of from about 4.01:1 to about 5:1, and provides methods of using the dosage forms of EPA/DHA to treat a variety of cardiovascular, autoimmune, inflammatory, and central nervous system disorders by administering a formulation of the invention to a patient in need thereof.

In some embodiments, the disclosure provides a composition of omega-3 fatty acids concentrates containing EPA and DHA, cannabanoids, and L-theanine, wherein the EPA to DHA ratio is not particular limited. For example, the EPA to DHA ratio may be present in the range of about 3.5-4.0:1, about 4.0-4.5:1, about 4.5-5.0:1, about 5.0-5.5:1, about 5.5-6.0:1, about 6.0-6.5:1, about 6.5-7.0:1, about 7.0-8.0:1, about 9.0-10.0:1 in the composition. In some specific embodiment, the composition contains essentially pure EPA with less than 4 wt % of DHA. Note that the method in produce omega-3 fatty acids concentrates is not particular limited, and it may include for example, supercritical and super fluid chromatography, urea precipitation, molecular distillation, and/or extraction. In a specific embodiment, the EPA to DHA ratio is 3.5-7:1 EPA/DHA and is prepared from marine lipids: predominately sourced from Peruvian anchovies and sardines. The marine lipids are processed into 18/12 or crude oil, followed by using supercritical and super fluid chromatography to break down the oil into constituent parts, and then reconstituted into 3.5-7:1 EPA/DHA ratio.

In some embodiments, the disclosure provides a composition containing 30-99 wt % omega-3 fatty acids concentrates, phytocannabanoids, and L-theanine. The wt % of omega-3 fatty acids in the concentrates is not particular limited. For example, the composition may contain omega-3 fatty acids concentrates with about 30-35 wt %, about 35-40 wt %, about 40-45 wt %, about 45-50 wt %, about 50-55 wt %, about 55-60 wt %, about 60-65 wt %, about 65-70 wt %, about 70-75 wt %, about 75-80 wt %, about 80-85 wt %, about 85-90 wt %, about 90-95 wt %, about 95-99 wt % omega-3 fatty acids.

In another embodiment, the disclosure provides a composition containing 30-99 wt % omega-3 fatty acids concentrates with EPA and DHA, phytocannabanoids, and L-theanine, wherein the EPA to DHA ratio is in the range of about 3.5-7.0:1.

In yet another embodiment, the disclosure provides a composition containing 30-99 wt % omega-3 fatty acids concentrates with EPA and DHA, phytocannabanoids, and L-theanine, wherein the EPA to DHA ratio is in the range of 4.01-5:1.

In some embodiments, the disclosure provides a composition containing omega-3 fatty acids concentrates with EPA and DHA, phytocannabanoids, and L-theanine, wherein the phytocannabanoids is full spectrum phytocannabinoids. Note that the method in producing full spectrum phytocannabinoids is not particular limited, and it may include for example, supercritical and super fluid chromatography, urea precipitation, molecular distillation, and/or extraction. In some specific embodiment, wherein the full spectrum phytocannabinoids are harvested from industrial hemp in foreign countries, e.g., Belgium.

In another embodiment, the disclosure provides a composition containing omega-3 fatty acids concentrates with EPA and DHA, phytocannabanoids, and L-theanine, wherein the phytocannabanoids containing cannabidiol (CBD) with less than 0.3 wt % of tetrahydrocannabinol (THC). Note that the method in producing cannabidiol (CBD) with less than 0.3 wt % of tetrahydrocannabinol (THC) is not particular limited, and it may include for example, supercritical and super fluid chromatography, urea precipitation, molecular distillation, and/or extraction. In some embodiments, cannabidiol (CBD) are obtained from full spectrum phytocannabinoids. In some specific embodiment, wherein the full spectrum phytocannabinoids are harvested from industrial hemp in foreign countries, e.g., Belgium. In another specific embodiment, the buds and flower parts are removed, and the stalks and stems are ground into a powder; that powder is then compressed into pellets, followed by super fluid chromatography to remove the tetrahydrocannabinol and reconstitute the pellets into cannabidiol oil.

In some embodiments, the amount of omega-3 fatty acids concentrates in the composition is in the amount of 100-2,000 mg, and is not particular limited. For example, the omega-3 fatty acids concentrates may be present in the amount of about 100-150 mg, about 150-200 mg, about 200-250 mg, about 250-300 mg, about 300-350 mg, about 350-400 mg, about 400-450 mg, about 450-500 mg, about 500-600 mg, about 600-700 mg, about 700-800 mg, about 800-1,000 mg, about 1,000-1,200 mg, about 1,200-1,400 mg, about 1,400-1,700 mg, about 1,700-2,000 mg in the composition.

In some embodiments, the phytocannabanoids in the composition is in the amount of 1-50 mg, and is not particular limited. For example, the phytocannabanoids may be present in the amount of about 1-2 mg, about 2-4 mg, about 4-6 mg, about 6-8 mg, about 8-10 mg, about 10-12 mg, about 12-16 mg, about 16-20 mg, about 20-25 mg, about 25-30 mg, about 30-35 mg, about 35-40 mg, about 40-45 mg, about 45-50 mg, about 50-55 mg, about 55-60 mg in the composition.

In some embodiments, the L-theanine in the composition is in the amount of 25-500 mg, and is not particular limited. For example, the L-theanine may be present in the amount of about 25-35 mg, about 35-45 mg, about 45-55 mg, about 55-65 mg, about 65-75 mg, about 75-100 mg, about 100-125 mg, about 125-150 mg, about 150-175 mg, about 175-200 mg, about 200-250 mg, about 250-300 mg, about 300-350 mg, about 350-400 mg, about 400-450 mg, about 450-500 mg in the composition.

In yet another embodiment, the disclosure provides a composition comprising 100-2,000 mg 30-99 wt % of omega-3 fatty acids concentrates, 1-50 mg phytocannabanoids, and 25-500 mg L-theanine. In some specific embodiment, the disclosure provides a composition comprising 100-350 mg of omega-3 fatty acids concentrates, 1-4 mg phytocannabanoids, and 25-99 mg L-theanine. In another specific embodiment, the disclosure provides a composition comprising 250-500 mg 50-70 wt % of omega-3 fatty acids concentrates, 2-8 mg phytocannabanoids, and 75-150 mg L-theanine. In yet another specific embodiment, the disclosure provides a composition comprising 350-2,000 mg 60-99 wt % of omega-3 fatty acids concentrates, 6-50 mg phytocannabanoids, and 125-500 mg L-theanine.

The present disclosure further provides the use of the compositions in addressing multiple indications for health and wellness, including but not limited to, sleeplessness, anxiety, pain associated with general inflammation, drug addiction, brain injury, and/or alpha wave brain disorder.

In another aspect, the description provides a composition containing the material produced from the methods described herein.

In another aspect, the description provides capsule, tablet, liquid, syrup, suspensions, sublingual, candy, and chewable dosage forms of the compositions described herein.

In another aspect, the compositions described herein are pharmaceutical compositions. In yet another aspect, the compositions described herein are dietary supplement compositions.

In yet another aspect, the pharmaceutical compositions further include an excipient. Excipients are selected with respect to the intended form of administration, e.g. oral tablets, capsules, powders, syrups, suspensions, and the like, and consistent with conventional pharmaceutical practices. For example, for oral administration in the form of gel capsule the formulation may be combined with a preservative, flavorant, colorant or the like.

In another aspect, the compositions described herein may include a stabilizer. “Stabilizers” include compounds which maintain a desirable attribute of the formulation over a time interval including but not limited to mechanical, chemical and temperature stressing that can be tested in a laboratory setting. Such attributes include stabilizing homogeneity resulting in concentrations consistent with the labeled potency, maintaining specified purity and dispersibility in simulated gastric and intestinal fluids without significant degradation of the attributes for which the stabilizer was employed. In some embodiments the stabilizer is an antioxidant, such as vitamin E. Other suitable antioxidants include hydroxytoluene, butyrate, quinone, ascorbic acid.

In another aspect, the compositions described herein may contain a preservative. Preservatives are compounds that inhibit microbial growth and are typically added to dispersions to prevent microbes from growing. Typically amounts of preservatives needed to pass anti-microbial effectiveness testing as described by USP and EU methodology are used to test appropriate preservative levels. Preservatives include but are not limited to potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

### EXAMPLES

The embodiments described above in addition to other embodiments can be further understood with reference to the following examples:

### Example 1

The composition is prepared by mixing the omega-3 fatty acids concentrates, full spectrum phytocannabinoids and L-Theanine together based on following formulations:

Formulation 1

| | | |
|---|---|---|
| Omega 3 (30-50% Concentrates) | 100-350 | mg |
| Full Spectrum Phytocannabinoids | 1-4 | mg |
| L-Theanine | 25-99 | mg |

Formulation 2

| | | |
|---|---|---|
| Omega 3 (50-70% Concentrates) | 250-500 | mg |
| Full Spectrum Phytocannabinoids | 2-8 | mg |
| L-Theanine | 75-150 | mg |

Formulation 3

| | | |
|---|---|---|
| Omega 3 (60-99% Concentrates) | 350-2000 | mg |
| Full Spectrum Phytocannabanoids | 6-50 | mg |
| L-Theanine | 125-500 | mg |

### Example 2: Clinical Study of the Composition of the Present Disclosure

The primary objective of this study is to ascertain the effects of the composition of the present disclosure on multiple markers of sleep. Secondary outcomes include changes in stress, relaxation, mood, C-reactive protein, cortisol, and clinical markers of safety [i.e. systemic hemodynamics (BP/HR)], side effect profiles/adverse event incidence and tolerance to supplementation.

### SUMMARY

This is a randomized, placebo-controlled, double-blind, parallel groups trial in 50 apparently healthy men and women.

N=50 apparently healthy volunteers will consume 2 soft gels of the composition of the present disclosure or a placebo for two weeks. Each soft gel contain about contain 100 mg of nutrient rich hemp oil (which is about 10 mg of full spectrum phytocannabanoids), about 500 mg of EPA/DHA omega-3 fatty acids, and about 100 mg of purified L-Theanine.

Measurements of sleep quality, stress, relaxation, and mood will be made prior to supplementation (baseline), and then again after 3 and 14 days of supplementation.

Subjects:

50 apparently healthy subjects between the ages of 25 and 60 years old.

All subjects will be pre-screened using health history questionnaires, vital signs, and blood work.

In the event a subject drops-out, he/she may be replaced. Subjects that drop out for safety reasons will not be replaced.

Estimated Study Duration:

Approximately 8-10 weeks, including screening and study duration.

A final report for the study will be provided to the sponsor approximately one month after completion of data collection (i.e. after QC and data analyses have been completed).

Inclusion/Exclusion Criteria:

Participants must meet all of the following study criteria in order to participate in the study:

Inclusion Criteria:

Provide voluntary signed and dated informed consent.

Be in good health as determined by medical history and routine blood chemistries.

Age between 25 and 60 years (inclusive).

Body Mass Index of 18.5-34.99 (inclusive).

Normotensive (supine, resting systolic blood pressure ≤140 mm Hg and diastolic blood pressure ≤90 mm Hg. If the first measurement is slightly elevated above these limits, the subject will be given a brief (5 minute) rest period, and two more measurements will be taken. The average of all three measurements will be used to determine eligibility.

Normal supine, resting heart rate (≤90 per minute).

Willing to duplicate their previous 24-hour diet, and fast for 10 hours prior to baseline, day 3 and day 14 visit.

Subjects who voluntarily report intermittent sleep problems (e.g. difficulty falling asleep, waking up often during the night and having trouble going back to sleep, having unrefreshing sleep, daytime sleepiness, etc) but have not been diagnosed with any specific sleep disorder. Additional information on sleep disorder classification may be found, for example, at the website of American Sleep Apnea Association.

Exclusion Criteria:

History of diabetes or stroke.

History of a breathing related sleep disorder, circadian rhythm sleep disorder, or dyssomnia not otherwise specified.

History of a sleep disorder because of a general medical condition,

History of psychiatric or neurological disorders (anxiety, depression, dementia) or use of any medications that affected the central nervous system or sleep/wake function within 2 weeks prior to the first day of the study.

Use of psychotropic treatments (neuroleptics, antiepileptics, barbiturates, antidepressants, anxiolytics, or lithium) in the previous 3 months.

History of malignancy in the previous 5 years except for non-melanoma skin cancer (basal cell cancer or squamous cell cancer of the skin).

Gastrointestinal or metabolic diseases that might impact nutrient absorption e.g. short bowel syndrome, diarrheal illnesses, history of colon resection, gastro paresis, Inborn-Errors-of-Metabolism (such as PKU).

Chronic inflammatory condition or disease (e.g. rheumatoid arthritis, Crohn's disease, ulcerative colitis, Lupus, HIV/AIDS, etc.).

Known sensitivity to any ingredient in the test formulations as listed in the Certificates-of-Analysis.

Currently participating in another research study with an investigational product or have been in another research study in the past 30 days.

Women currently breastfeeding a child.

Women with a positive pregnancy test.

Any other diseases or conditions that, in the opinion of the medical staff, could confound the primary endpoint or place the subject at increased risk of harm if they were to participate.

Birth Control:

Females of child-bearing potential must not be pregnant and not get pregnant while in the study. Abstinence or a barrier type of birth control (e.g., condom with spermicide, diaphragm with spermicide) must be employed.

Diet:

All subjects will be asked to maintain their current dietary habits.

Each subject's baseline diet (24-hour diet record) will be analyzed via NutriBase IX (Clinical Edition) to determine its energy and macronutrient content.

To replicate baseline-testing conditions as closely as possible, subjects will follow their previously recorded 24-hour diet records, and fast for 10 hours prior to day 3 and day 14 visits.

In addition, 3-day diet records will be collected prior to week 0 and again prior to day 14.

Physical Activity:

Subjects will be asked to maintain their current levels of habitual physical activity during the study (documented via the Framingham Physical Activity questionnaire).

Supplement:

Subjects will consume the manufacturer's suggested dose of product (2 soft gels prior to sleep).

Blood Samples:

There will be two blood draws during the screening process. The blood draws will be performed by trained study staff using a needle stick in your arm. The total amount of blood drawn at each visit will be about 16 mL (2 tablespoons). For comparison, the standard blood donation is about 480 mL (two cups).

Adverse Events:

During each visit, all local and systemic non-serious and serious adverse events (AEs) observed by or reported by the investigators will be evaluated and assessed through reports coded using the Medical Dictionary for Regulatory Activities (MedDRA) and Common Terminology Criteria for Adverse Events (CTCAE) for AE term and organ system class. The intensity of an AE will be graded according to the protocol-defined toxicity criteria based on the 2009 DAIDS Therapeutic Research Program's “Table for Grading Severity of Adult Adverse Experiences” (see page 10 for details).

Study Procedures

Visit 1 (Screening)

Medical history, routine safety blood work (CBC, CMP, and Lipid Panel), and baseline diet. Urinary pregnancy test for females. Remind subjects not to exercise for 24 hours prior to reporting to the laboratory.

Visit 2 (Week 0)—Collect the Following Information:

Body weight, body mass index, vital signs (HR and BP).

Blood sample for C-reactive protein and cortisol.

PSQI (Pittsburgh Sleep Quality Index) validated questionnaire instrument to assess sleep quality, disruption and patterns in adults over the 2-week interval (See Buysse D J, Reynolds C F, Monk T H, Berman S R, Kupfer D J. The Pittsburgh Sleep Quality Index: a new instrument for psychiatric practice and research. Psychiatr Res 1989; 28:193-213).

Stanford Sleepiness Scale, which is a quick way to assess how alert you are feeling. If it is during the day when you go about your business, ideally you would want a rating of a one. Take into account that most people have two peak times of alertness daily, at about 9 a.m. and 9 p.m. Alertness wanes to its lowest point at around 3 p.m.; after that it begins to build again. Rate your alertness at different times during the day. If you go below a three when you should be feeling alert, this is an indication that you have a serious sleep debt and you need more sleep. Stanford Sleepiness Scale has the following scale ratings:

| Degree of Sleepiness | Scale Rating |
|---|---|
| Feeling active, vital, alert, or wide awake | 1 |
| Functioning at high levels, but not at peak; able to | 2 |
| concentrate | |
| Awake, but relaxed; responsive but not fully alert | 3 |
| Somewhat foggy, let down | 4 |
| Foggy; losing interest in remaining awake; slowed | 5 |
| down | |
| Sleepy, woozy, fighting sleep; prefer to lie down | 6 |
| No longer fighting sleep, sleep onset soon; having | 7 |
| dream-like thoughts | |
| Asleep | X |

Epworth Sleepiness Scale (ESS), which was introduced at the Epworth Hospital in Melbourne in 1990 by Dr Murray Johns, which focuses on measuring the Average Sleep Propensity (ASP) in selected types of day to day activities. ASP has three stages of different severity: mild, moderate and severe, that are used to quantify the propensity of falling asleep.

Anchored Visual Analog Scales (VAS) for feelings of relaxation, mental clarity, positive mood, perceived feelings of stress, sleep and well-being.

Framingham Activity questionnaire.

AE monitoring/ask subjects if they have had a change in their health and/or medication status since their last visit.

Visit 3 (Day 3):

Body weight, body mass index, vital signs (HR and BP).

PSQI (Pittsburgh Sleep Quality Index) validated questionnaire instrument to assess sleep quality, disruption and patterns in adults over the 2-week interval.

Stanford Sleepiness Scale.

Epworth Sleepiness Scale.

Anchored Visual Analog Scales (VAS) for feelings of relaxation, mental clarity, positive mood, perceived feelings of stress, sleep and well-being.

Framingham Activity questionnaire

AE monitoring/ask subjects if they have had a change in their health and/or medication status since their last visit.

Visit 4 (Day 14):

Body weight, body mass index, vital signs (HR and BP).

Blood sample for C-reactive protein and cortisol.

PSQI (Pittsburgh Sleep Quality Index) validated questionnaire instrument to assess sleep quality, disruption and patterns in adults over the 2-week interval.

Stanford Sleepiness Scale.

Epworth Sleepiness Scale.

Anchored Visual Analog Scales (VAS) for feelings of relaxation, mental clarity, positive mood, perceived feelings of stress, sleep and well-being.

Framingham Activity questionnaire

AE monitoring/ask subjects if they have had a change in their health and/or medication status since their last visit.

Methods:

To replicate baseline testing conditions as closely as possible, subjects will follow their previously recorded 24-hr diet records, refrain from exercise for 24 hours, and fast for 10-hours prior to all laboratory visits.

The experimental procedures used in the study will be communicated to subjects prior to enrollment.

Health history will be determined using a standardized questionnaire.

Heart rate and blood pressure will be measured using an automated sphygmomanometer.

Standing height will be determined using a wall-mounted stadiometer.

Body weight will be measured using a Seca™ Medical Scale.

LabCorp (Dublin, Ohio) will be utilized to transport and analyze all blood samples.

Comprehensive clinical chemistries, hematology, and lipid profile will be analyzed at screening.

Diet records will be analyzed using Nutribase IX Nutrition Software, CyberSoft, Inc. (Phoenix, Ariz.).

Study Schematic

| | | Visit 2 | Visit 3 | Visit 4 |
|---|---|---|---|---|
| PROCEDURE | Screening | (Day 0) | (Day 3) | (Day 14) |
| Informed Consent | X | | | |
| Inclusion/Exclusion Criteria | X | | | |
| Medical History | X | | | |
| Hgt, weight, & vitals (BP | X | X | X | X |
| and HR) | | | | |
| Safety Screen (CMP, CBC, | X | | | |
| lipid panel) | | | | |
| Pittsburgh Sleep Quality | | X | X | X |
| Index | | | | |
| Stanford Sleepiness Scale | | X | X | X |
| Epworth Sleepiness Scale | | X | X | X |
| Blood sample (C-reactive | | X | | X |
| protein and cortisol) | | | | |
| (VAS) for feelings of | | X | X | X |
| relaxation, mental | | | | |
| clarity, positive mood, | | | | |
| perceived feelings of | | | | |
| stress, sleep and | | | | |
| well-being | | | | |
| Diet Records/Analysis/ | | X | X | X |
| Background Diet* | | | | |
| Physical Activity | | X | X | X |
| Questionnaire | | | | |
| Protocol Compliance | | X | X | X |
| Dispense Test Product | | X | | |
| Adverse Event Monitoring | | X | X | X |
| Product Side Effect | | X | X | X |
| Questionnaire | | | | |

*3-day diet records conducted/collected at screening visit and prior to day 14. Food frequency questionnaires and/or 24-hr recalls will be assessed during visit 3 and 4.

Statistical Analyses:

Outcome variables will be displayed via line/bar graphs (plotting mean+/−SD) to visually compare responses between groups. ANOVA (on change scores) and/or ANCOVA will be used to ascertain treatment effects. Within-group effects will be determined with paired t-tests. Significance will be set at P<0.05 and trends defined as 0.051<P<0.10. Effect sizes will also be calculated. The research team may consult statistical advisors and consider their input/assistance on data handling and statistical analyses.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

What is claimed is:

1. A composition comprising
omega-3 fatty acids concentrates,
phytocannabanoids, and
L-theanine.

2. The composition according to claim 1, wherein the omega-3 fatty acids concentrates contains 30-99 wt % omega-3 fatty acids.

3. The composition according to claim 2, wherein the omega-3 fatty acids concentrates contains EPA and DHA in a ratio of 3.5-7:1.

4. The composition according to claim 3, wherein the omega-3 fatty acids concentrates contains EPA and DHA in a ratio of 4.01-5:1.

5. The composition according to claim 1, wherein the omega-3 fatty acids concentrates is in the amount of 100-2,000 mg.

6. The composition according to claim 1, wherein the phytocannabanoids is full spectrum phytocannabinoids.

7. The composition according to claim 1, wherein the phytocannabanoids comprises cannabidiol (CBD) with less than 0.3 wt % of tetrahydrocannabinol (THC).

8. The composition according to claim 7, wherein the phytocannabanoids is produced by removing the buds and flower parts;
grounding the stalks and stems are ground into a powder;
compressing the powder into pellets;
removing tetrahydrocannabinol (THC) by super fluid chromatography; and
reconstituting the pellets into cannabidiol oil (CBD).

9. The composition according to claim 1, wherein the phytocannabanoids is in the amount of 1-50 mg.

10. The composition according to claim 1, wherein the L-theanine is in the amount of 25-500 mg.

11. A composition comprises
100-2,000 mg 30-99 wt % of omega-3 fatty acids concentrates,
1-50 mg phytocannabanoids, and
25-500 mg L-theanine.

12. The composition according to claim 11 comprises
100-350 mg 30-50 wt % of omega-3 fatty acids concentrates,
1-4 mg phytocannabanoids, and
25-99 mg L-theanine.

13. The composition according to claim 11 comprises
250-500 mg 50-70 wt % of omega-3 fatty acids concentrates,
2-8 mg phytocannabanoids, and
75-150 mg L-theanine.

14. The composition according to claim 11 comprises
350-2,000 mg 60-99 wt % of omega-3 fatty acids concentrates,
6-50 mg phytocannabanoids, and
125-500 mg L-theanine.

15. The composition according to claim 11, wherein
the omega-3 fatty acids concentrates comprises EPA and DHA in a ratio of 3.5-7:1; and
the phytocannabanoids comprises cannabidiol (CBD) with less than 0.3 wt % of tetrahydrocannabinol (THC).

16. A method of using the composition according to claim 1 comprises administering the composition in a human with sleeplessness, anxiety, pain associated with general inflammation, drug addiction, brain injury, and/or alpha wave brain disorder.

17. A method of using the composition according to claim 15 comprises administering the composition in a human with sleeplessness, anxiety, pain associated with general inflammation, drug addiction, brain injury, and/or alpha wave brain disorder.

18. A composition comprises
400-600 mg 30-99 wt % of omega-3 fatty acids concentrates,
8-12 mg phytocannabanoids, and
75-125 mg L-theanine.

19. The composition according to claim 18, wherein the omega-3 fatty acids concentrates contains 30-99 wt % omega-3 fatty acids, and the omega-3 fatty acids concentrates contains EPA and DHA in a ratio of about 4:1.

20. A method of using the composition according to claim 18 comprises administering the composition in a human with sleeplessness and anxiety.
